(19) United States
(12) Patent Application Publication
(10) Pub. No.: US 2019/0091272 A1
(43) Pub. Date: Mar. 28, 2019

(54) ANTI-CANCER DRUG COMPOSITION INCLUDING GANODERMA EXTRACT AND AMPHOTERICIN B

(71) Applicants: Ching-Hua Su, Taipei City (TW); Cheng-Jeng Tai, Taipei City (TW); Taiwan Indigena Botanica Co., Ltd., Taipei City (TW)

(72) Inventors: Ching-Hua Su, Taipei City (TW); Cheng-Jeng Tai, Taipei City (TW)

(21) Appl. No.: 16/200,344

(22) Filed: Nov. 26, 2018

Related U.S. Application Data
(62) Division of application No. 15/044,794, filed on Feb. 16, 2016.

(30) Foreign Application Priority Data
Aug. 25, 2015 (TW) 104127785

(51) Int. Cl. A61K 36/074; A61K 31/7048

(52) U.S. Cl. CPC A61K 36/074; A61K 31/7048; A61K 2300/00

(57) ABSTRACT

An anti-cancer drug composition includes a Ganoderma extract having a concentration of 1-5 mg/ml and an amphotericin B having a concentration of 3-10 μM. The Ganoderma extract and the amphotericin B are medicated at different times by pre-treating cancer cells with the Ganoderma extract for a period of time followed by administration of the amphotericin B to enhance an anti-cancer effect of the amphotericin B.

### CROSS-REFERENCE TO RELATED APPLICATION

This application is a Divisional Application of U.S. patent application Ser. No. 15/044,794 filed on Feb. 16, 2016 and entitled “ANTI-CANCER DRUG COMPOSITION INCLUDING GANODERMA EXTRACT AND AMPHOTERICIN B”, which claims priority to Taiwan patent application No. 104127785 filed on Aug. 25, 2015 and entitled “ANTI-CANCER DRUG COMPOSITION INCLUDING GANODERMA EXTRACT AND AMPHOTERICIN B”, the entirety of which is hereby incorporated by reference.

### FIELD OF THE INVENTION

The present invention relates to an anti-cancer drug composition, and more particularly to an anti-cancer drug composition including Ganoderma extract and amphotericin B.

### BACKGROUND OF THE INVENTION

According to the statistics of recent years, malignant tumors occupy the first position of the ten leading causes of death in Taiwan, wherein the lung cancer, the liver cancer and the colorectal cancer are the top three death-causing cancers in Taiwan. Therefore, how to effectively cure cancers and reduce the death rates of the cancers is an important issue in the medical field.

Due to the rapid development of civilization, there exist many invisible harmful substances in the environment, and it is harmful to human bodies after exposing in such environment for a long time. By epidemiology and statistics studies, it is shown that about 80% of the cancers are caused by external factors, including changes of lifestyle, infections caused by infectious diseases, and occupations and the environments thereof. Particularly, the chemical carcinogens are the most dangerous factors. Once the chemical carcinogen is absorbed by the human body and further metabolized and activated, it may cause a series of mutations to the intracellular genes. As a result, the cells may be out of control and keep dividing and growing, and finally the tumor cells are formed.

Many kinds of cancer therapies, such as chemotherapy, radiotherapy, immunotherapy, monoclonal antibody therapy and gene therapy, are developed. However, many problems are also resulted from the therapies. For example, a series of side effects are caused by the chemotherapy, or some patients may have resistance to the drugs.

On the other hand, some cancer therapies focus on natural foods to select the functional or nutrient foods having anti-cancer potential. In the previous studies, several Chinese medicinal herbs are presumed to have anti-cancer effect. In the present invention, the active ingredients of lingzhi mushroom are extracted, and their synergistic effect with amphotericin B is further studied to develop an anti-cancer drug composition having more curative effect.

### SUMMARY OF THE INVENTION

An object of the present invention is to study the synergistic effect of lingzhi extracts and amphotericin B to develop an anti-cancer drug composition having more curative effect.

According to an aspect of the present invention, there is provided a an anti-cancer drug composition including a Ganoderma extract having a concentration of 1-5 mg/ml and an amphotericin B having a concentration of 3-10 μM. The Ganoderma extract and the amphotericin B are medicated at different times by pre-treating cancer cells with the Ganoderma extract for a period of time followed by administration of the amphotericin B to enhance an anti-cancer effect of the amphotericin B.

In an embodiment, the Ganoderma extract is extracted from Ganoderma lucidum, and particularly extracted from fruiting bodies of Ganoderma lucidum.

In an embodiment, the Ganoderma extract is a Ganoderma ethanolic extract.

In an embodiment, the Ganoderma extract includes sterols and triterpenoids.

In an embodiment, the amphotericin B has a concentration of 3-5 μM.

In an embodiment, the period of time is 12-48 hours.

In an embodiment, the period of time is 24 hours.

In an embodiment, the anti-cancer drug composition is used to treat liver cancers.

In an embodiment, the anti-cancer drug composition is used to treat lung cancers.

The above objects and advantages of the present invention will become more readily apparent to those ordinarily skilled in the art after reviewing the following detailed description and accompanying drawings, in which:

### BRIEF DESCRIPTION OF THE DRAWINGS

FIGS. 1A and 1B show the results for Hep G2 cells of Example 1 in the pre-treatment group and the co-treatment group, respectively;

FIGS. 1C and 1D show the results for Hep 3B cells of Example 1 in the pre-treatment group and the co-treatment group, respectively;

FIGS. 1E and 1F show the results for Hep 5J cells of Example 1 in the pre-treatment group and the co-treatment group, respectively;

FIGS. 2A and 2B show the results for Hep G2 cells of Example 2 in the pre-treatment group and the co-treatment group, respectively;

FIGS. 2C and 2D show the results for Hep 3B cells of Example 2 in the pre-treatment group and the co-treatment group, respectively;

FIGS. 2E and 2F show the results for Hep 5J cells of Example 2 in the pre-treatment group and the co-treatment group, respectively;

FIG. 3A shows the results for A549 cells of Example 3; and

FIG. 3B shows the results for H460 cells of Example 3.

### DETAILED DESCRIPTION OF THE PREFERRED EMBODIMENT

The present invention will now be described more specifically with reference to the following embodiments. It is to be noted that the following descriptions of preferred embodiments of this invention are presented herein for purpose of illustration and description only; it is not intended to be exhaustive or to be limited to the precise form disclosed.

According to the previous studies, some medicinal fungus contains abundant triterpenoids, polysaccharides and sterols, which have certain curative effects in protecting livers, promoting liver cell regeneration, and anti-liver cancer. In addition, amphotericin B (also referred as AmB) is a polyene antifungal agent used to treat fungal infection by direct binding to ergosterol on fungal membrane, which induces pore formation on fungal membrane and alters cell membrane permeability, and thus leads to cell death. Therefore, the present invention further studies the synergistic effect of the lingzhi extracts and the amphotericin B to develop an anti-cancer drug composition having more curative effect.

The present invention aims to study if the lingzhi extracts can enhance the anti-cancer effect of the amphotericin B to inhibit the growth of liver cancer cells. The lingzhi mushrooms used in the present invention were Ganoderma lucidum, and were extracted with ethanol to obtain the Ganoderma ethanolic extracts. The fruiting bodies of Ganoderma lucidum were first sliced and powdered. Then 10 grams of the Ganoderma powder was added into 500 ml of 99% ethanol and stirred for 24 hours at 200 rpm. The solution was further filtered to collect the filtrate, and the filtrate was concentrated under reduced pressure to obtain the dry extract powder, which was further weighted to calculate the recovery rate and then stored at 4° C. in dark place.

Certainly, the Ganoderma extracts are not limited to Ganoderma ethanolic extracts. Other extraction methods, which are capable of extracting sterols and triterpenoids from Ganoderma, and the extracts thereof are also applicable to this invention. For example, the organic solvent (such as n-Hexane) having different polarity may also be used for extraction, but not limited thereto.

The following experiments are used to further describe the examples of the anti-cancer drug compositions of the present invention.

### Example 1

This example used human liver carcinoma cell lines including Hep G2, Hep 3G and Hep 5J for experiments, and the cells were cultured in 96-well plates. The experiments were divided into two groups including a pre-treatment group and a co-treatment group. The cells in the pre-treatment group were pre-treated with the Ganoderma ethanolic extracts for 24 hours and then treated with the amphotericin B for 48 hours, while the cells in the co-treatment group were co-treated with the Ganoderma ethanolic extracts and the amphotericin B together for 48 hours.

In the pre-treatment group, the cells were pre-treated with the Ganoderma ethanolic extracts at concentrations of 0, 1, 2.5 and 5 mg/ml for 24 hours, and then treated with the amphotericin B at concentrations between 0-80 μM for 48 hours, followed by MTT assay. In the co-treatment group, the cells were co-treated with the Ganoderma ethanolic extracts at concentrations of 0, 1, 2.5 and 5 mg/ml and the amphotericin B at concentrations between 0-80 μM for 48 hours, followed by MTT assay. The MTT solution (3-(4,5-Dimethylthiazol-2-yl)-2,5-diphenyltetrazolium bromide) was added to each well, and the plates were further incubated for 45-60 minutes. The cell culture medium was then removed and replaced by DMSO (dimethyl sulfoxide). Subsequently, the optical density (OD) was measured at 550 nm.

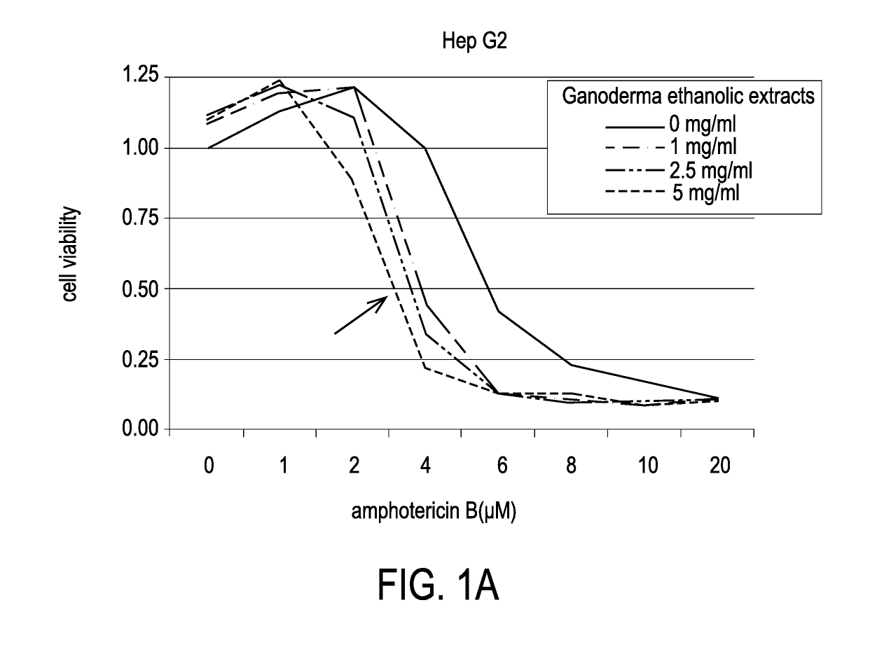

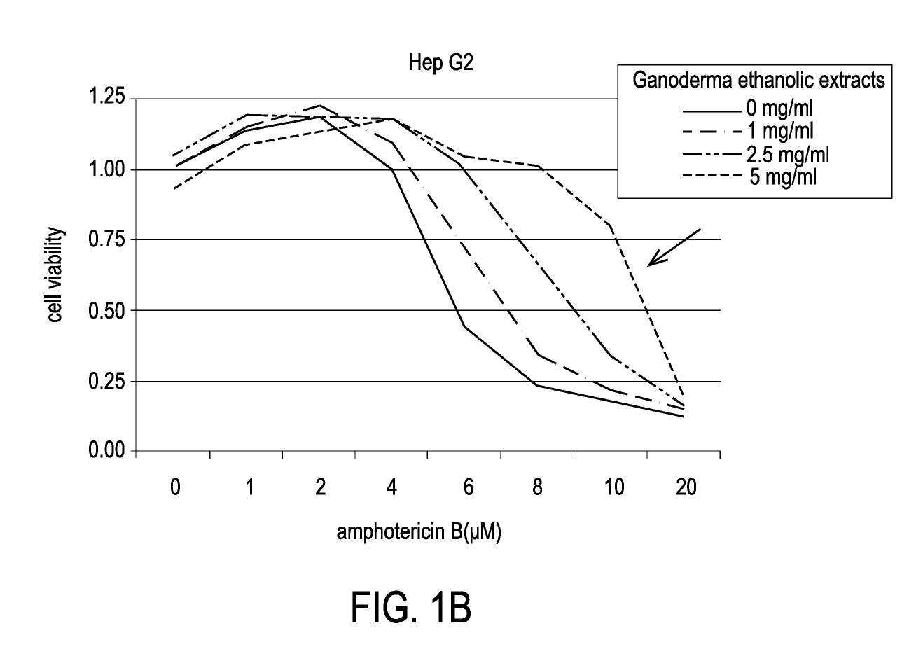

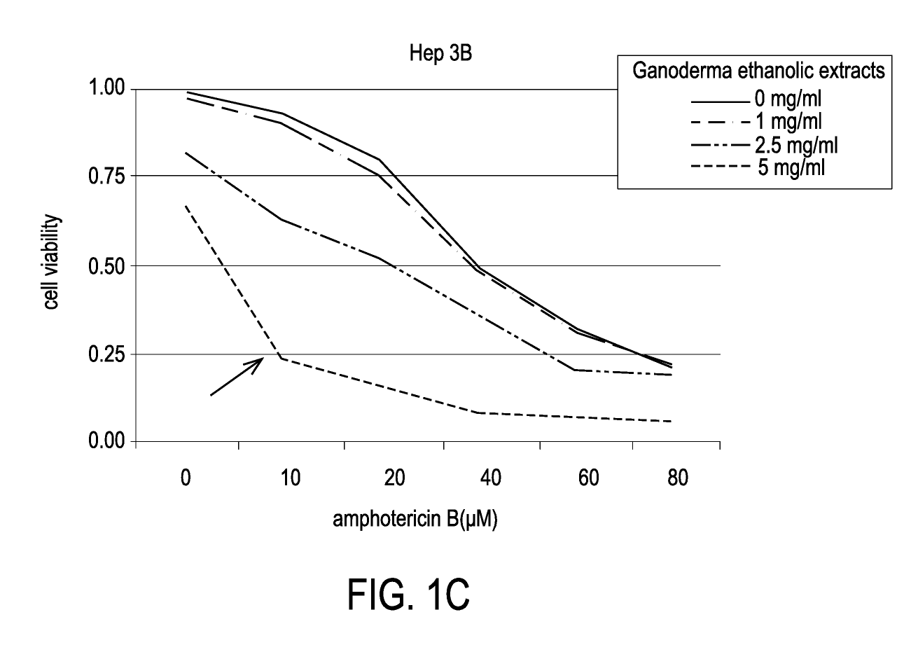

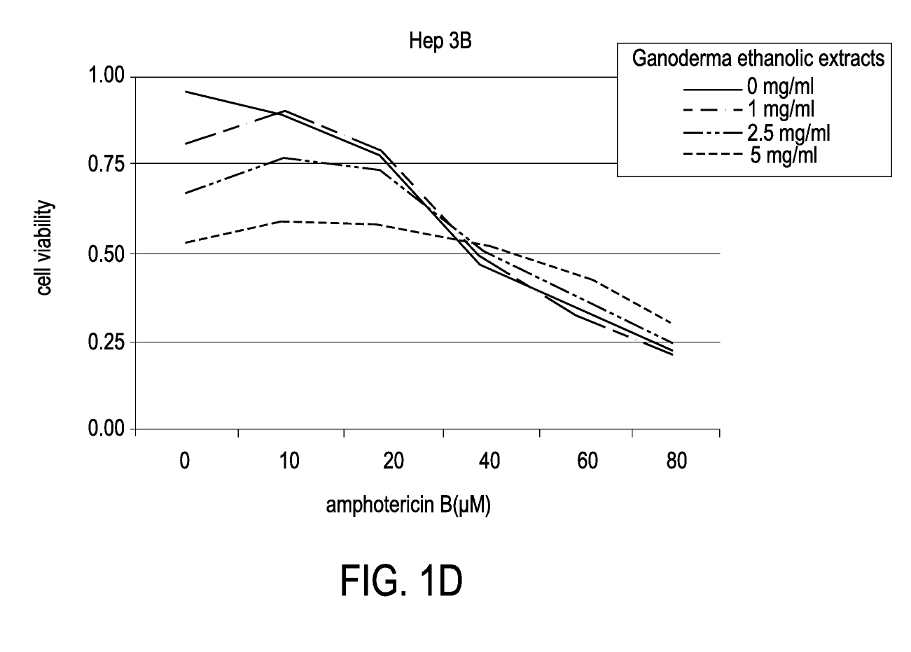

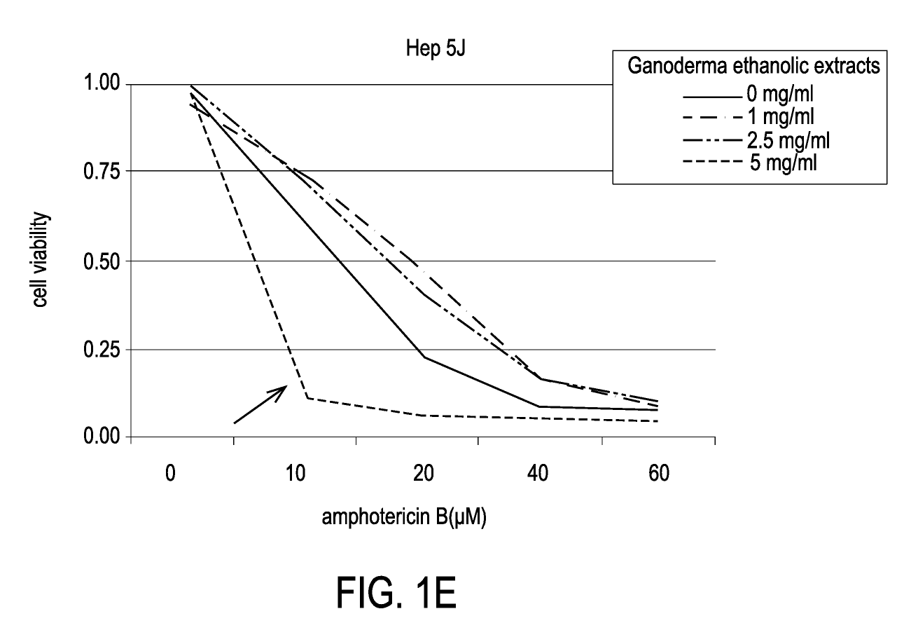

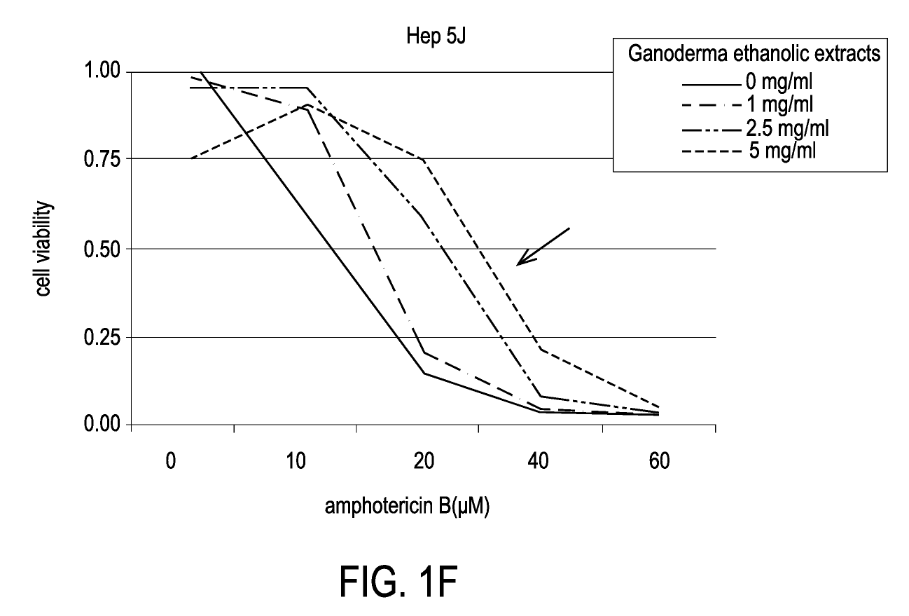

FIGS. 1A and 1B show the results for Hep G2 cells of Example 1 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 1A show that, compared with the line for 0 mg/ml of Ganoderma ethanolic extract (i.e. the cells were only treated with the amphotericin B), pre-treatments with the Ganoderma ethanolic extracts at concentrations of 1, 2.5 and 5 mg/ml significantly enhanced the inhibitory effects of the amphotericin B on Hep G2 cells, and when the drug dose of the amphotericin B was 5 μM, about 75% cell growth was inhibited, which showed very good anti-cancer effect. However, the results of FIG. 1B show that co-treatments with 1, 2.5 and 5 mg/ml of Ganoderma ethanolic extracts and the amphotericin B significantly antagonized the inhibitory effects of the amphotericin B on Hep G2 cells, and the antagonistic ability was proportional to the dose of the Ganoderma ethanolic extract.

FIGS. 1C and 1D show the results for Hep 3B cells of Example 1 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 1C show that, compared with the line for 0 mg/ml of Ganoderma ethanolic extract (i.e. the cells were only treated with the amphotericin B), pre-treatments with the Ganoderma ethanolic extracts at concentrations of 2.5 and 5 mg/ml significantly enhanced the inhibitory effects of the amphotericin B on Hep 3B cells, and the inhibitory effect was proportional to the dose of the Ganoderma ethanolic extract. When the dose of the Ganoderma ethanolic extract was 5 mg/ml and the dose of the amphotericin B was 10 μM, about 75% cell growth was inhibited, which showed very good anti-cancer effect. However, from FIG. 1D, co-treatments with the Ganoderma ethanolic extracts and the amphotericin B did not show significant effect.

FIGS. 1E and 1F show the results for Hep 5J cells of Example 1 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 1E show that, compared with the line for 0 mg/ml of Ganoderma ethanolic extract (i.e. the cells were only treated with the amphotericin B), pre-treatment with the Ganoderma ethanolic extract at concentration of 5 mg/ml significantly enhanced the inhibitory effect of the amphotericin B on Hep 5J cells, and when the dose of the Ganoderma ethanolic extract was 5 mg/ml and the dose of the amphotericin B was 10 μM, about 85% cell growth was inhibited, which showed very good anti-cancer effect. Besides, pre-treatments with the Ganoderma ethanolic extracts at concentrations of 1 and 2.5 mg/ml slightly antagonized the inhibitory effects of the amphotericin B on Hep 5J cells. Further, the results of FIG. 1F show that co-treatments with 1, 2.5 and 5 mg/ml of Ganoderma ethanolic extracts and the amphotericin B significantly antagonized the inhibitory effects of the amphotericin B on Hep 5J cells, and the antagonistic ability was proportional to the dose of the Ganoderma ethanolic extract.

From the above results of Example 1, it is clear that pre-treatments with the Ganoderma ethanolic extracts for 24 hours significantly enhanced the anti-cancer effect of the amphotericin B on the three human liver carcinoma cell lines including Hep G2, Hep 3G and Hep 5J, but the enhancements did not exist in the co-treatment groups, and the co-treatments even antagonized the anti-cancer effect of the amphotericin B. Therefore, it is concluded that adequately using the Ganoderma ethanolic extracts, such as pre-treating the cancer cells with the Ganoderma ethanolic extracts in a sufficient dose for a period of time, can significantly enhance the anti-cancer effect of the amphotericin B on the human liver carcinoma cells, so the Ganoderma ethanolic extracts and the amphotericin B which are medicated at different times can be developed to be an anti-cancer drug composition having more curative effect.

### Example 2

The experiment methods in Example 2 were the same as those in Example 1, but using different batches of cells.

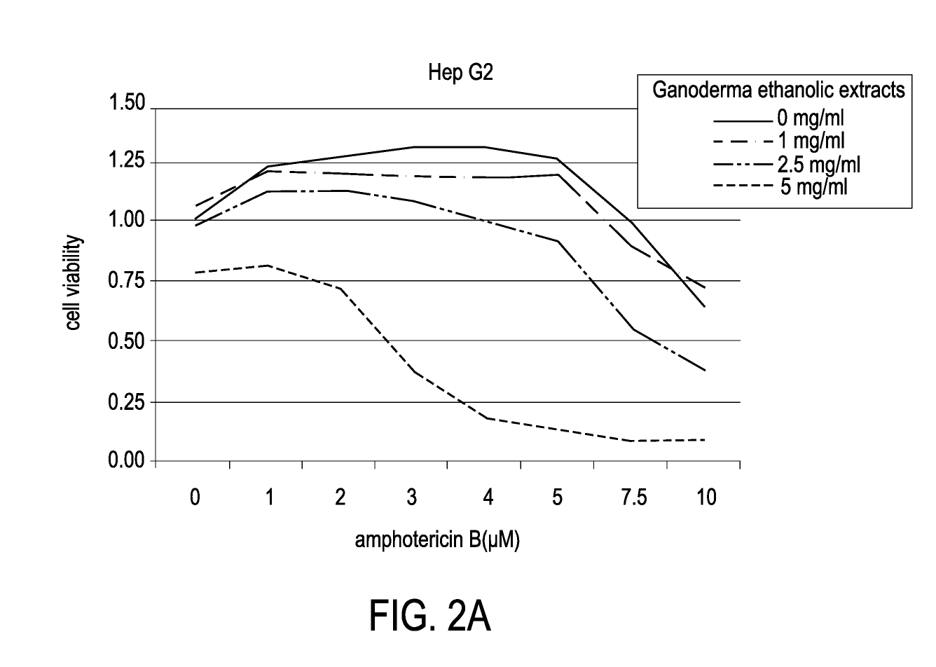

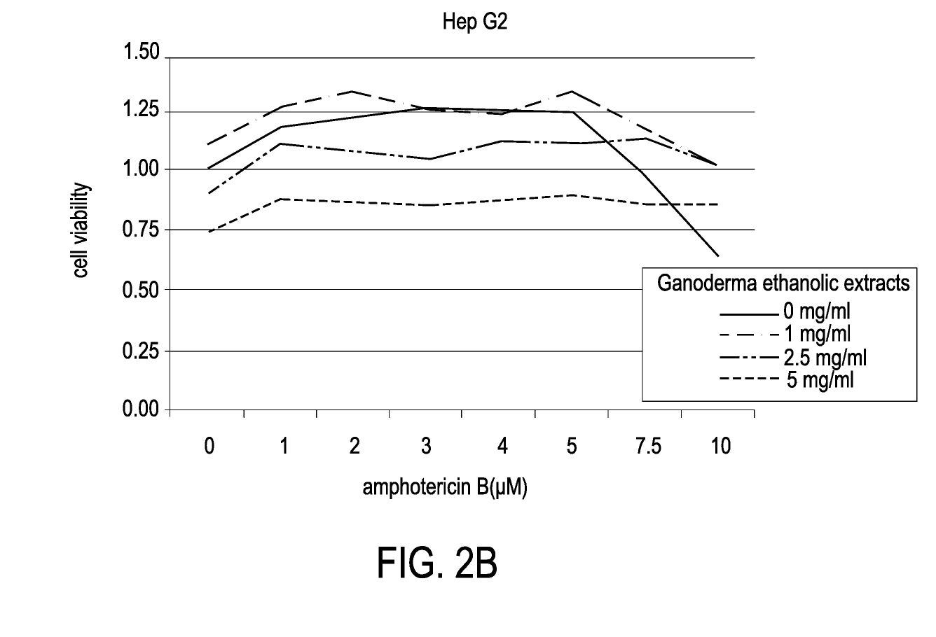

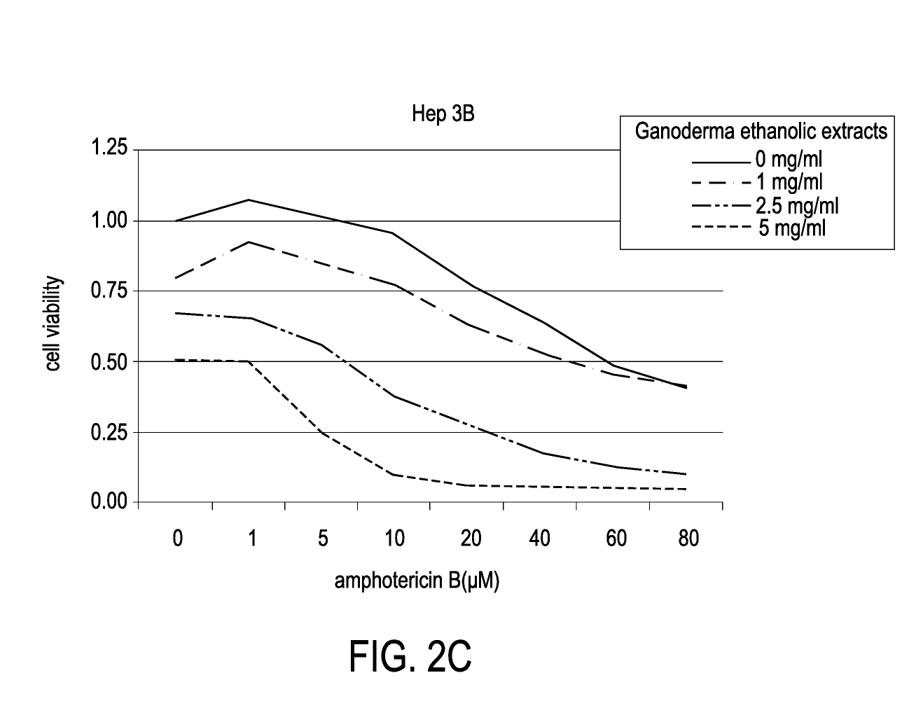

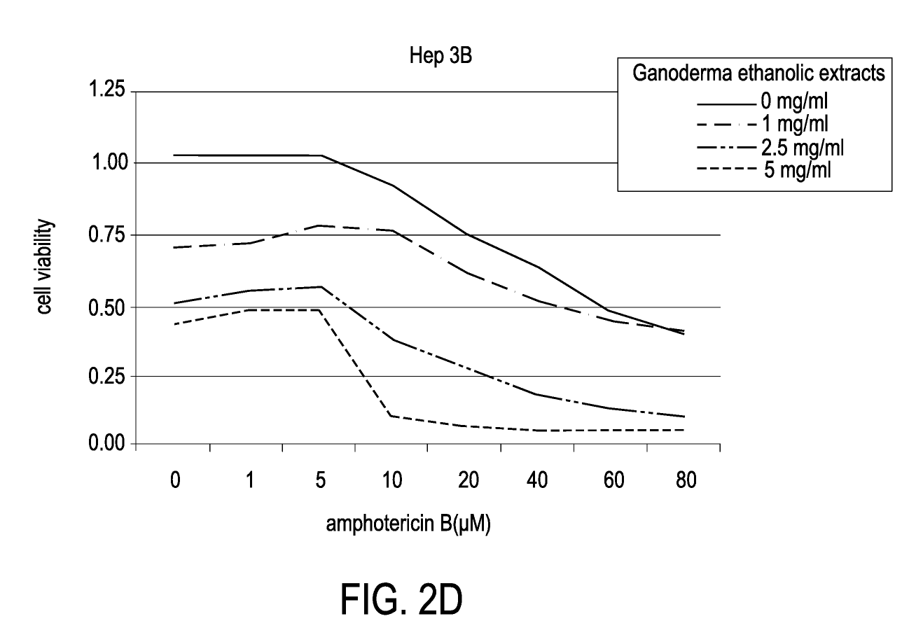

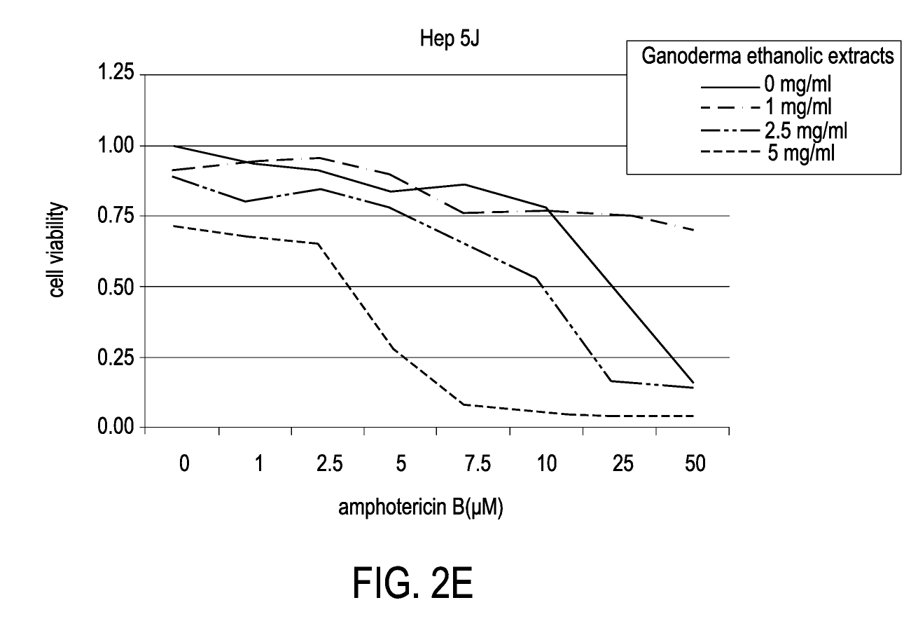

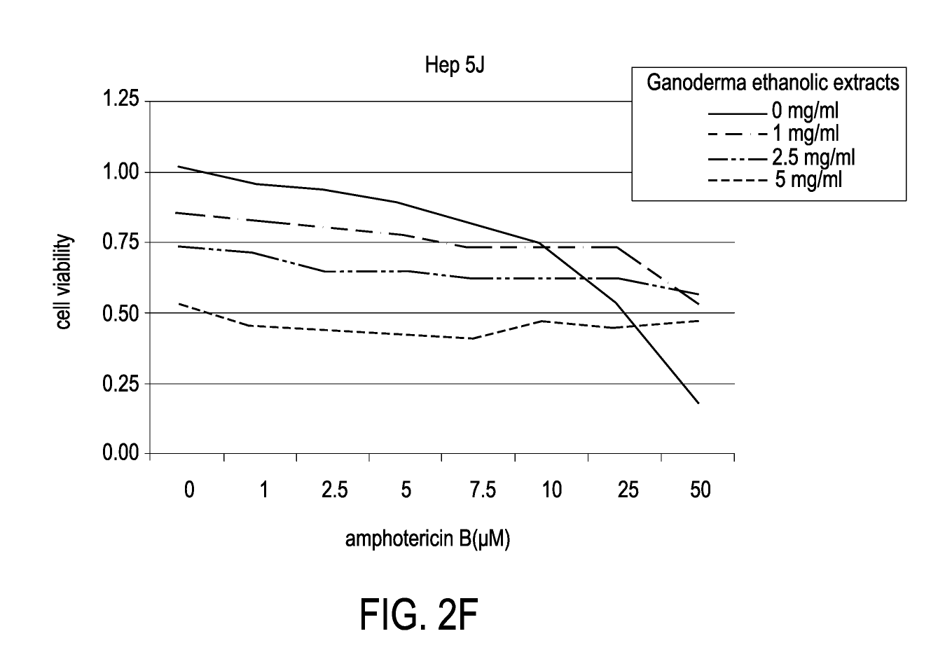

FIGS. 2A and 2B show the results for Hep G2 cells of Example 2 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 2A show that, compared with the line for 0 mg/ml of Ganoderma ethanolic extract (i.e. the cells were only treated with the amphotericin B), pre-treatments with the Ganoderma ethanolic extracts at concentrations of 1, 2.5 and 5 mg/ml significantly enhanced the inhibitory effects of the amphotericin B on Hep G2 cells, and the inhibitory effect was proportional to the dose of the Ganoderma ethanolic extract. When the dose of the Ganoderma ethanolic extract was 5 mg/ml and the dose of the amphotericin B was 4 μM, about 80% cell growth was inhibited, which showed very good anti-cancer effect. However, from FIG. 2B, co-treatments with 1, 2.5 and 5 mg/ml of Ganoderma ethanolic extracts and the amphotericin B antagonized the inhibitory effects of the amphotericin B on Hep G2 cells instead.

FIGS. 2C and 2D show the results for Hep 3B cells of Example 2 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 2C show that, compared with the line for 0 mg/ml of Ganoderma ethanolic extract (i.e. the cells were only treated with the amphotericin B), pre-treatments with the Ganoderma ethanolic extracts at concentrations of 1, 2.5 and 5 mg/ml significantly enhanced the inhibitory effects of the amphotericin B on Hep 3B cells, and the inhibitory effect was proportional to the dose of the Ganoderma ethanolic extract. When the dose of the Ganoderma ethanolic extract was 5 mg/ml and the dose of the amphotericin B was 5 μM, about 75% cell growth was inhibited, which showed very good anti-cancer effect. Besides, the results of FIG. 2D show that co-treatments with the Ganoderma ethanolic extracts and the amphotericin B also significantly enhanced the inhibitory effects of the amphotericin B on Hep 3B cells.

FIGS. 2E and 2F show the results for Hep 5J cells of Example 2 in the pre-treatment group and the co-treatment group, respectively, wherein the horizontal axis indicates the concentration of the amphotericin B, and the vertical axis indicates the cell viability. The four lines represent the concentrations of the Ganoderma ethanolic extracts at 0, 1, 2.5 and 5 mg/ml, respectively. The results of FIG. 2E show that, compared with the line for 0 mg/ml of Ganoderma ethanolic extract (i.e. the cells were only treated with the amphotericin B), pre-treatments with the Ganoderma ethanolic extracts at concentrations of 2.5 and 5 mg/ml significantly enhanced the inhibitory effects of the amphotericin B on Hep 5J cells, and when the dose of the Ganoderma ethanolic extract was 5 mg/ml and the dose of the amphotericin B was 5 μM, about 75% cell growth was inhibited, which showed very good anti-cancer effect. However, pre-treatment with the Ganoderma ethanolic extract at concentration of 1 mg/ml slightly antagonized the inhibitory effect of the amphotericin B on Hep 5J cells. Further, from FIG. 2F, co-treatments with the Ganoderma ethanolic extracts and the amphotericin B antagonized the inhibitory effects of the amphotericin B on Hep 5J cells instead.

From the above results of Example 2, it is clear that pre-treatments with the Ganoderma ethanolic extracts for 24 hours significantly enhanced the anti-cancer effect of the amphotericin B on the three human liver carcinoma cell lines including Hep G2, Hep 3G and Hep 5J, which are similar to the results of Example 1.

According to the examples of the present invention, pre-treatments with the Ganoderma ethanolic extracts can significantly enhance the anti-cancer effect of the amphotericin B, wherein the effective dose of the Ganoderma ethanolic extract is 1-5 mg/ml, and the effective dose of the amphotericin B is at least 3 μM. However, if the dose of the amphotericin B is too high, serious side effects may be resulted, so the clinical dose of the amphotericin B has to be carefully controlled. Based on the results from Examples 1 and 2, the safe and effective dose of the amphotericin B is 3-10 μM, and is preferably 3-5 μM. In addition, the pre-treatment period of time for the Ganoderma ethanolic extract is not limited to 24 hours, and it may also be 12-48 hours.

The principle of why pre-treatments with the Ganoderma ethanolic extracts can significantly enhance the anti-cancer effect of the amphotericin B is supposed as follows. The sterols in the cell membranes of cancer cells are different from the sterols in the cell membranes of normal cells, and have more unsaturated double bonds, and thus, the cancer cells are softer and easier to move and transfer, and this phenomenon does not exist in the normal cells. Since the Ganoderma ethanolic extracts contain fungal sterols and triterpenoids, and the fungal sterols have more double bonds when compared with the sterols of the normal cells, the fungal sterols of the Ganoderma ethanolic extracts are easy to replace the sterols in the cell membranes of cancer cells during the rapid replication and mitosis processes. Moreover, the fungal sterols having more double bonds show greater affinity to the amphotericin B, so the amphotericin B is easy to bind with the fungal sterols in the cell membranes of cancer cells, which induces pore formation, alters cell membrane permeability and thus leads to cell death. Therefore, pre-treatments with the Ganoderma ethanolic extract may enhance the sensitivity of cancer cells toward cell membrane destruction induced by the amphotericin B, and thus can be developed to be the anti-cancer drug composition used for targeted therapy to cancer cells. Based on this mechanism, it is believed the anti-cancer drug composition of the present invention is not limited to treat liver cancers, and should also be used to treat other cancers, such as lung cancers and colorectal cancers.

The following example is to further test and verify if the anti-cancer drug composition of the present invention can also be used to treat other cancers.

### Example 3

This example used human lung cancer cell lines including A549 and H460 for experiments, and the cells were cultured in 96-well plates. The cells were pre-treated with the Ganoderma ethanolic extract at concentration of 2.5 mg/ml for 24 hours, and then treated with the amphotericin B for 48 hours.

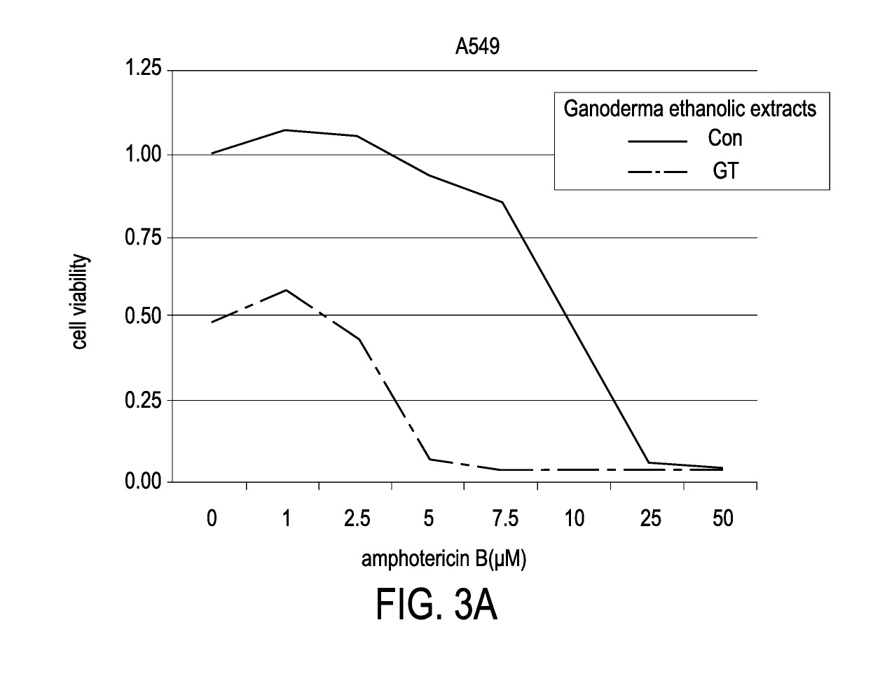

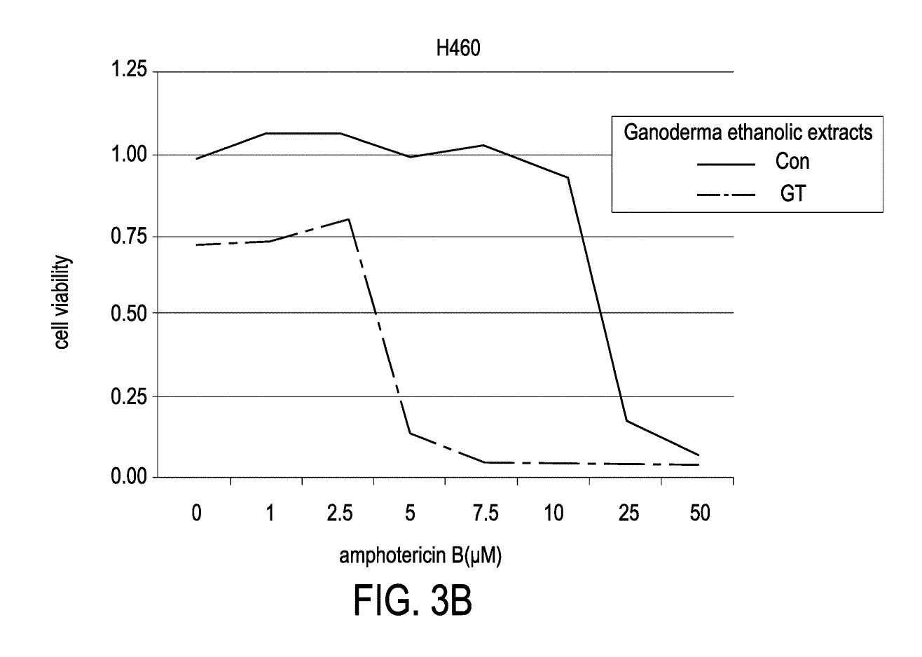

FIG. 3A shows the results for A549 cells of Example 3, wherein the horizontal axis indicates the concentration of the amphotericin B, and the vertical axis indicates the cell viability. The line Con represents no pre-treatment with the Ganoderma ethanolic extract, and the line GT represents pre-treatment with the Ganoderma ethanolic extract at concentration of 2.5 mg/ml. The results of FIG. 3A show that, compared with the line Con (i.e. the cells were only treated with the amphotericin B), pre-treatment with the Ganoderma ethanolic extract at concentration of 2.5 mg/ml significantly enhanced the inhibitory effect of the amphotericin B on A549 cells, and when the drug dose of the amphotericin B was 5 μM, about 90% cell growth was inhibited, which showed very good anti-cancer effect.

FIG. 3B shows the results for H460 cells of Example 3, wherein the horizontal axis indicates the concentration of the amphotericin B, and the vertical axis indicates the cell viability. The line Con represents no pre-treatment with the Ganoderma ethanolic extract, and the line GT represents pre-treatment with the Ganoderma ethanolic extract at concentration of 2.5 mg/ml. The results of FIG. 3B show that, compared with the line Con (i.e. the cells were only treated with the amphotericin B), pre-treatment with the Ganoderma ethanolic extract at concentration of 2.5 mg/ml significantly enhanced the inhibitory effects of the amphotericin B on H460 cells, and when the drug dose of the amphotericin B was 5 μM, about 85% cell growth was inhibited, which showed very good anti-cancer effect.

In conclusion, the present invention provides an anti-cancer drug composition including the Ganoderma extract and the amphotericin B, wherein the cancer cells are pre-treated with the Ganoderma extract for a period of time and then treated with the amphotericin B, and it is proved that pre-treatments with the Ganoderma extract can significantly enhance the anti-cancer effect of the amphotericin B. In other words, the Ganoderma extract and the amphotericin B are medicated at different times by pre-treating cancer cells with the Ganoderma extract followed by administration of the amphotericin B, so as to enhance the anti-cancer effect of the amphotericin B. Since the anti-cancer drug composition of the present invention can inhibit at least 75% cancer cell growth and reduce the drug dose of the amphotericin B, the present invention possesses high medical value.

While the invention has been described in terms of what is presently considered to be the most practical and preferred embodiments, it is to be understood that the invention needs not be limited to the disclosed embodiment. On the contrary, it is intended to cover various modifications and similar arrangements included within the spirit and scope of the appended claims which are to be accorded with the broadest interpretation so as to encompass all such modifications and similar structures.

What is claimed is:

1. A method of treating cancers with an anti-cancer drug composition including a Ganoderma ethanolic extract and an amphotericin B, the method comprising pre-treating cancer cells with the Ganoderma ethanolic extract for a period of time followed by administration of the amphotericin B to enhance an anti-cancer effect of the amphotericin B, wherein the Ganoderma ethanolic extract has a concentration of 1-5 mg/ml, the amphotericin B has a concentration of 3-10 μM, the period of time is 12-48 hours, and the anti-cancer effect of the amphotericin B is proportional to the concentration of the Ganoderma ethanolic extract.

2. The method according to claim 1 wherein the Ganoderma ethanolic extract is extracted from Ganoderma lucidum.

3. The method according to claim 1 wherein the Ganoderma ethanolic extract is extracted from fruiting bodies of Ganoderma lucidum.

4. The method according to claim 1 wherein the Ganoderma ethanolic extract includes sterols and triterpenoids.

5. The method according to claim 1 wherein the amphotericin B has a concentration of 3-5 μM.

6. The method according to claim 1 wherein the period of time is 24 hours.

7. The method according to claim 1 being used to treat liver cancers.

8. The method according to claim 1 being used to treat lung cancers.
